(19) United States
(12) Patent Application Publication
(10) Pub. No.: US 2015/0110666 A1
(43) Pub. Date: Apr. 23, 2015

(54) ACETONE STORAGE

(71) Applicant: BOREALIS AG, Vienna (AT)

(72) Inventors: René Dicke, Leonding (AT); Andreas Leitner, Freistdadt (AT)

(21) Appl. No.: 14/381,571

(22) PCT Filed: Feb. 26, 2013
(86) PCT No.: PCT/EP2013/053789

§ 371 Date: Aug. 27, 2014

(30) Foreign Application Priority Data
Feb. 28, 2012 (EP) 12157255.6

(51) Int. Cl. F17C 3/12; F17D 1/08; C22C 38/02; C22C 38/58; C22C 38/44; C22C 38/04; C07C 49/08; F17C 3/02

(52) U.S. Cl. CPC F17C 3/12; C07C 49/08; F17D 1/08; F17C 3/02; C22C 38/58; C22C 38/44; C22C 38/04; C22C 38/02
USPC 420/52; 568/383; 420/43; 420/67

(57) ABSTRACT

An acetone storage tank or acetone transfer pipe comprising stainless steel in which the amount of Cr is in the range 10.5 wt % to 20 wt %; the amount of Ni is ≧9 wt %, and the amount of Mo is 2.75%≧Mo≧0 wt %, of the stainless steel.

This invention relates to the storage of acetone and in particular to the storage of acetone at high temperatures which might be experienced in warmer climates. More specifically, the invention concerns the design of acetone storage tanks and transfer pipes whose make up can be tailored to prevent chemical reactions of the acetone within the tank or pipes, in particular in areas of high natural temperature.

Acetone is a widely used and hence widely stored organic chemical. It is well known that acetone decomposes by acid and base catalyzed Aldol-type reactions. Trace amounts of acid or base produce system inherent by-products like diacetone alcohol (DAA), mesityl oxide (MO) and oligomers even at ambient temperature.

A major use of acetone is in the formation of bisphenol A or as solvent in the pharmaceutical industry. For the use of acetone in bisphenol A production or as a solvent in the pharmaceutical industry the requirements for purification are very tight. Acetone with significant impurities might not fulfil the necessary specification after long term storage. Mesityloxide, in particular, is an unwanted impurity in acetone used for bisphenol A production.

During their research, the present inventors found that pure acetone from different sources significantly decomposes, at room temperature as well as elevated temperature, which causes a reduced shelf life and quality issues. Our results suggest that at elevated temperatures of 50° C., such as might be experienced in hot climates such as the middle East, the acetone specification limit can be passed after 2 months of storage.

A process to store acetone with less likelihood of decomposition is highly desirable. A second point is that acetone is a commodity product and cheap. Pure acetone must be generally available cheaply. The industry cannot afford to spend money purifying stored acetone before selling it to a customer. The storage process must result in acetone that is maintained within purity limits.

Acetone is often stored in stainless steel tanks. The present inventors suggest that one or more of the metal types in steel catalyses the aldol condensation reaction discussed above. This is a problem whether an acetone storage tank is located in an environment where high temperatures and strong sunshine can be expected such as in the middle east or in more temperate climates. The inventors sought a solution to this problem. In particular, the present inventors have realised that the nature of the storage tanks themselves is an important factor contributing to the stability of the stored acetone.

There is no apparent prior art on the relevance of the nature of the storage tank to the stability of the acetone within it. It is generally known that acetone can be stored within stainless steel tanks or Zn coated tanks but such tanks are generally suggested for the storage of most organic chemicals anyway.

Sunoco Chemicals suggest the use of carbon steels or stainless steels for acetone storage on their website. We show below that carbon steels are very poor materials for acetone storage. In the Dow “Answer section” of their website, Dow teach that acetone can be stored in unlined carbon steel tanks with a Zn lining being recommended for applications where purity is essential. Shell recommends the same materials for acetone storage as Dow. The large industry players all therefore propose roughly the same solution for acetone storage. No one before has considered whether the steels used can themselves influence the degradation of the acetone within. For the avoidance of doubt, in this invention, we regard stainless steel as a steel alloy with a minimum of 10.5% chromium content by mass.

In the article “Temporal stability of Polar Organic Compounds in Stainless Steel Canisters”, Journal of the Air and Waste Management Association, vol. 42, no. 4, April 1992, p. 460-462, the authors conclude that where chemicals such as acetone are stored in stainless steel tanks, it is best practice to minimise water content.

No one before has considered whether the make up of the steel might contribute to the degradation of the acetone within a tank. Steel is made up of many different metals and some of these may be more likely to catalyse degradation reactions than others.

The present inventors have found therefore that careful control of the steel used in acetone storage tanks can ensure that the speed of acetone degradation is minimised.

The present inventors have surprisingly found that where the make up of the steel meets the limits defined below in claim 1, the amounts of impurities formed on long term storage of acetone can be minimised. Moreover, the principles described herein in relation to acetone can also be applied to the storage of other aldehydes and ketones.

### SUMMARY OF INVENTION

Thus, viewed from one aspect the invention provides an acetone storage tank or acetone transfer pipe comprising stainless steel in which the amount of Cr is in the range 10.5 wt % to 20 wt %; the amount of Ni is 9 wt %, such as 9 to 15 wt % and the amount of Mo is 2.75 wt %≧Mo≧0 wt %, of the stainless steel.

Viewed from another aspect the invention provides an acetone storage tank or transfer pipe as hereinbefore defined containing acetone, preferably acetone and any impurities only.

Viewed from another aspect the invention provides use of stainless steel as an inhibitor of the formation of impurities during storing of an aldehyde or ketone such as acetone in which the amount of Cr is in the range 10.5 wt % to 20 wt %; the amount of Ni is ≧9 wt %, and the amount of Mo is 2.75%≧Mo≧0 wt %, of the stainless steel.

Viewed from another aspect the invention provides the use of stainless steel in which the amount of Cr is in the range 10.5 wt % to 20 wt %; the amount of Ni is 9 wt %, and the amount of Mo is 2.75%≧Mo≧0 wt %, of the stainless steel in the manufacture of an acetone storage tank or transfer pipe.

Viewed from another aspect the invention provides a process for reducing the formation of impurities such as diacetone alcohol in acetone comprising storing acetone in a tank or transfer pipe as hereinbefore defined.

### DETAILED DESCRIPTION OF INVENTION

This invention relates to the storage of aldehydes and ketones, in particular acetone. As we show clearly in the results below, the nature of the steel used to store acetone at various temperatures has a significant impact on the stability of the acetone within the storage tank.

The nature of the acetone stored in the tanks of the invention is not important although this will typically be of sufficient purity to be used in the formation of bisphenol A or be pharmaceutical grade. Before storage any acetone will be nominally pure. Thus, before storage therefore the levels of impurity within the acetone may be very low. In particular, the amounts of diacetone alcohol may be less than 80 ppm, especially less than 50 ppm, most especially less than 25 ppm The level of mesityl oxide is preferably below 5 ppm, especially below 1 ppm in the acetone before storage. Ideally, there is no detectable mesityl oxide in the acetone to be stored.

The term stored implies that the acetone spends a prolonged period within a tank or pipe, such as more than a week or even more than a month or more than two months.

The size of the storage tanks or transfer pipes of the invention is not important. The invention relates to the manufacture of very large stationary tanks, portable tanks or smaller tanks which might be used in research facilities. Also, the invention relates to transfer pipes which might transport acetone to/from a tank. It is likely that some acetone remains within a transfer pipe so it will be important to prevent chemical reactions taking place in transfer pipes. The size of these pipes is obviously governed by the amount of acetone being transferred. Pipe diameters of at least 5 cm are preferred.

The storage tanks of the invention may be any convenient size, such as up to 4000 m³ in size. They may hold from 100 L to 100,000 L therefore, preferably 1000 L to 50,000 L.

The tanks can be subject to overpressures such as up to 15 bar to suppress volatility. Some tanks may use an aluminium lid to lie on top of the acetone as is known.

Our results indicate that it is important to maintain a constant atmosphere to prevent mesityl oxide formation within the acetone. It is preferred therefore if the atmosphere within the tank or pipe is kept constant and water is not allowed into the tank/pipe e.g. via humidity in the air.

In general, the design of the storage tanks and pipes is conventional other than the nature of the steel used. Thus, the acetone inlets and outlets, any valves present, cooling jackets and so on which typically form part of any storage tank can be present in their conventional form. It will be clear that if these parts are exposed to acetone and if those parts are manufactured using stainless steel then the use of the steels advocated in this invention for those parts will be preferred.

It will be appreciated that tanks and pipes will be exposed to different levels of heat depending on the time of year and time of day but it is important to keep the temperature down at all times.

If the temperature can be kept to 25° C. or less at all times that is ideal and suppresses impurity formation. Tanks/pipes may therefore be provided with appropriate cooling mechanisms such as a cooling jacket.

In hot climates however, it simply may not be possible to keep temperatures to levels lower than 25° C. without prohibitively expensive cooling techniques and that is where the use of the steels of the invention is most valuable as the steels minimise impurity formation. It is a feature of the invention therefore that the storage tanks and pipes of the invention and specifically the acetone therein are intermittently subject to temperatures of at least 25° C.

It will be appreciated that at no point should the acetone be allowed to evaporate so its temperature is preferably kept below the boiling point of acetone under the conditions in the tank, typically therefore under 56° C. The acetone stored should be a liquid.

It is a feature of the invention that the storage tanks/pipes can be unlined. There are suggestions in the art that Zn lined tanks are good for storing purer acetone. Zn lined tanks are costly and by using the steels of the invention, their use is unnecessary. The storage tanks of the invention can therefore be free of any lining layer such as a Zn lining layer or epoxy layer.

The steels of the storage tanks of the invention should not be sulphided or oxidised in carbon dioxide at high temperature. The steels should not be exposed to hydrogen sulphide, thiophene, sulphur dioxide or COS.

### Steels

The invention primarily relates to the selection of particular steels for the formation of acetone storage tanks/pipes. Careful steel selection has been found to suppress impurity formation. It is preferred if the steel of the invention is a stainless steel. By definition therefore the steels of the invention comprise 10.5 wt % Cr as a minimum and up to 20 wt % Cr. Preferably, the amount of Cr is ≦18.5 wt %, and most preferable ≦17.5 wt % of the stainless steel. The lower limit is preferably at least 11 wt % such as at least 12 wt % even at least 13 wt % Cr.

Preferred ranges are therefore 11 to 20 wt %, preferably 12 to 18.5 wt % especially 13 to 17.5 wt % Cr.

The amount of Ni is ≧9 wt %, preferably ≧10%, more preferably ≧10.5%, and most preferably ≧11% of the stainless steel. The upper limit for Ni is preferably 15 wt % such as up to 14 wt %.

The amount of Mo is in the range of 2.75%≧Mo≧0 wt %. Thus, the steels of the invention can be free of Mo. Preferably, the steels of the invention contain 2.4 wt %≧Mo≧0.05 wt %, and most preferable 2.3%≧Mo≧0.5 wt % of the stainless steel. It is preferred if Mo is present.

The carbon content of the steels used to manufacture acetone storage tanks according to the invention may be less than 0.5 wt %, especially less than 0.1 wt %. An appropriate minimum value is 0.001 wt %. Whilst carbon should be present therefore, the level thereof is not thought to be significant in terms of the storage of acetone.

Stainless steels may also contain Si. The level of Si may be between 0.01 to 1 wt %. Whilst Si should be present, the level thereof is not thought to be significant in terms of the storage of acetone.

The steels of the invention may contain Mn. Levels of Mn may be between 0.1 and 2 wt %, preferably between 0.1 and 1.75 wt %.

Preferred steels comprise the above amounts of Mn, C and Si.

The steels of the invention may contain P. Levels of P may be at least 0.02 wt %. Typically there is no more than 0.5 wt % P.

The steels of the invention may contain S. Levels of S may be at least 0.02 wt %. Typically there is no more than 0.5 wt % S. Steels of the invention may also contain amounts of one or more of the following elements: Cu, Ti or Nb as dopants. The amounts of these elements might vary but preferably none forms more than 0.3 wt % of any steel of the invention.

All steels contain iron, typically at least 60 wt % iron, preferably at least 70 wt % iron.

It is preferred if the steels of the invention do not contain any metals other than Fe, Cr, Ni, Mo, Mn, Cu, Ti and Nb. The steels need not contain all of these metals but preferably no other metal outside this list is present. In this regard, C and Si are not metals.

Preferred steels consists of some or all of Fe, C, Si, Mn, Cr, Ni, P, S, Cu, Ti, Nb and Mo only. More preferred steels consists of some or all of Fe, C, Si, Mn, Cr, Ni and Mo only.

Particular steels of interest are 1.4306, 1.4541 and 1.4571 steels, preferably 1.4571. Generally therefore steels of interest have:

14 to 18.5 wt % Cr, 0 to 2.5 wt % Mo and 9 to 13.5 wt % Ni.

Steels suitable for use in the invention are widely commercially available.

### Impurities Formed

The most important impurities which are formed are diacetone alcohol (DAA) and further derivatives like mesityloxide (MO) and isomesityloxide (IMO). The formation of mesityloxide is more probable than the iso-isomer. This chemistry is summarised in scheme 1:

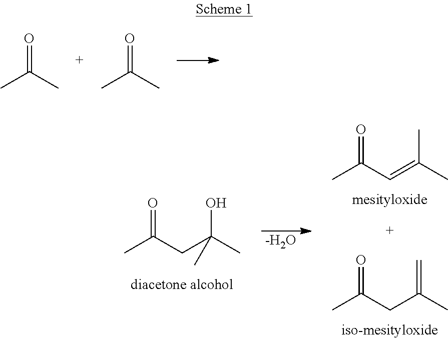

The tanks and pipes of the invention allow the amounts of these impurities to be minimised during storage. At any time therefore, it is preferred if the amount of DAA within an acetone containing storage tank or pipe of the invention is less than 80 ppm, especially less than 50 ppm, preferably less than 40 ppm, especially less than 20 ppm, more especially less than 10 ppm.

It is also preferred if the level of MO is less than 5 ppm, especially less than 1 ppm at any time.

It is preferred if the total amount of impurities within the tank or pipe is below 300 ppm at any time, preferably below 200 ppm. These amounts can be determined by GC analysis well known in the art.

It is preferred if the level of DAA within the acetone in a storage tank of the invention after storage at 25° C. for 60 days is less than 50 ppm, preferably less than 40 ppm, especially less than 20 ppm, more especially less than 11 ppm.

It is preferred if the level of DAA within the acetone in a storage tank of the invention after storage at 25° C. for 90 days is less than 50 ppm, preferably less than 40 ppm, especially less than 20 ppm, more especially less than 11 ppm. It is preferred if the level of DAA within the acetone in a storage tank of the invention after storage at 25° C. for 120 days is less than 50 ppm, preferably less than 40 ppm, especially less than 20 ppm.

It is preferred if the level of DAA within the acetone in a storage tank of the invention after storage at 50° C. for 60 days is less than 80 ppm, preferably less than 70 ppm, especially less than 60 ppm.

These values are attained preferably where the level of DAA in the acetone before storage was approximately 10 ppm or less.

It is preferred if the level of MO within the acetone in a storage tank of the invention after storage at 25° C. for 60 days is less than 5 ppm, preferably less than 2 ppm, especially less than 1 ppm.

It is preferred if the level of MO within the acetone in a storage tank of the invention after storage at 25° C. for 90 days is less than 5 ppm, preferably less than 2 ppm, especially less than 1 ppm.

It is preferred if the level of MO within the acetone in a storage tank of the invention after storage at 50° C. for 60 days is less than 5 ppm, preferably less than 4 ppm, especially less than 3 ppm. These values are attained preferably where the level of MO in the acetone before storage was less than 1 ppm.

It is preferred that the level of any of all impurities in the stored acetone is after 60 days storage in the tanks of the invention at 25° C. is less than 200 ppm, preferably less than 190 ppm.

The total impurity level in acetone after 60 days storage in the tanks of the invention at 50° C. should be less than 375 ppm, preferably less than 350 ppm.

It is also preferred if the number of different impurities within a tank or pipe is less than 15, especially less than 11.

It is also believed that the steel can act as an inhibitor of the auto-oligomerisation of acetone. Thus, viewed from another aspect the invention provides a process for inhibiting the formation of impurities in acetone comprising storing acetone in a tank as hereinbefore defined, in particular, a process for inhibiting the formation of acetone oligomers and their dehydration products (e.g. diacetone alcohol, mesityl oxide) in acetone comprising storing acetone in a tank as hereinbefore defined.

Viewed from another aspect the invention provides a process for storing acetone comprising putting acetone having a diacetone alcohol content of less than 25 ppm and/or a mesityl oxide content of less than 1 ppm in a stainless steel storage tank/pipe as hereinbefore defined;

storing said acetone within the tank/pipe for a period of at least one month during which time said tank or pipe is repeatedly exposed to a temperature of at least 25° C. but preferably no more than 50° C. such that the amount of diacetone alcohol in the acetone after one month is below 80 ppm and/or that the amount of mesityl oxide in the acetone after one month is below 5 ppm.

Viewed from another aspect the invention provides a process for storing acetone comprising putting acetone having a diacetone alcohol content of less than 25 ppm and/or a mesityl oxide content of less than 1 ppm in a stainless steel storage tank/pipe as hereinbefore defined;

storing said acetone within the tank/pipe for a period of at least two months during which time said tank or pipe is repeatedly exposed to a temperature of at least 25° C. but preferably no more than 50° C. such that the amount of diacetone alcohol in the acetone after two months is below 80 ppm and/or that the amount of mesityl oxide in the acetone after two months is below 5 ppm.

Whilst the present invention is described in connection with acetone, it is believed that the steels advocated for use in this invention are also of use in the storage of other aldehydes and ketones, such as methylethyl ketone. Other aldehydes and ketones of interest are low molecular weight compounds, typically of molecular weight of less than 200 g/mol.

Thus viewed from another aspect the invention provides a storage tank or transfer pipe for an aldehyde or ketone comprising stainless steel in which the amount of Cr is in the range 10.5 wt % to 20 wt %; the amount of Ni is 9 wt %, and the amount of Mo is 2.75%≧Mo≧0%, of the stainless steel. In particular, the tank should contain an aldehyde or ketone. The embodiments described herein in relation to acetone are applicable to other aldehydes and ketones.

It will be appreciated that the tanks/pipes of the invention will store exclusively acetone or exclusively other aldehydes or ketones. Thus the aldehyde or ketone will form the only material stored within the tank/pipe along of course with any impurities. The liquid within the tank or pipe will preferably be at least 99 wt % aldehyde or ketone, especially acetone. Other aldehydes and ketones of interest are typically of low molecular weight, e.g. less than 200 g/mol.

The invention will now be described with reference to the following non limiting examples and figures.

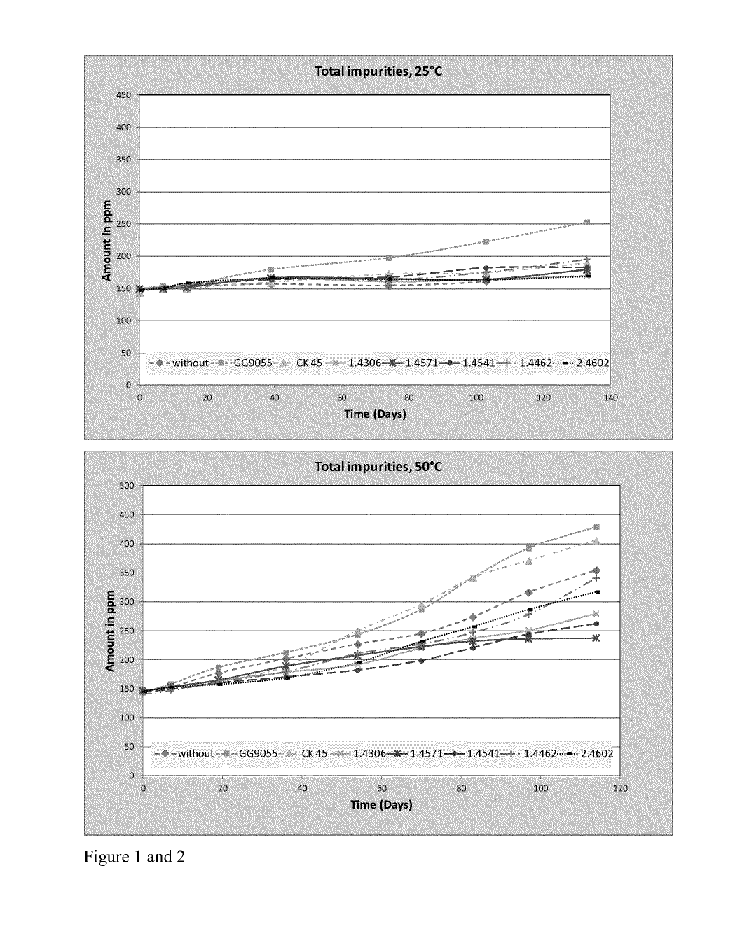

FIG. 1 is an illustration of total impurities vs time at 25° C.

FIG. 2 is an illustration of total impurities vs time at 50° C.

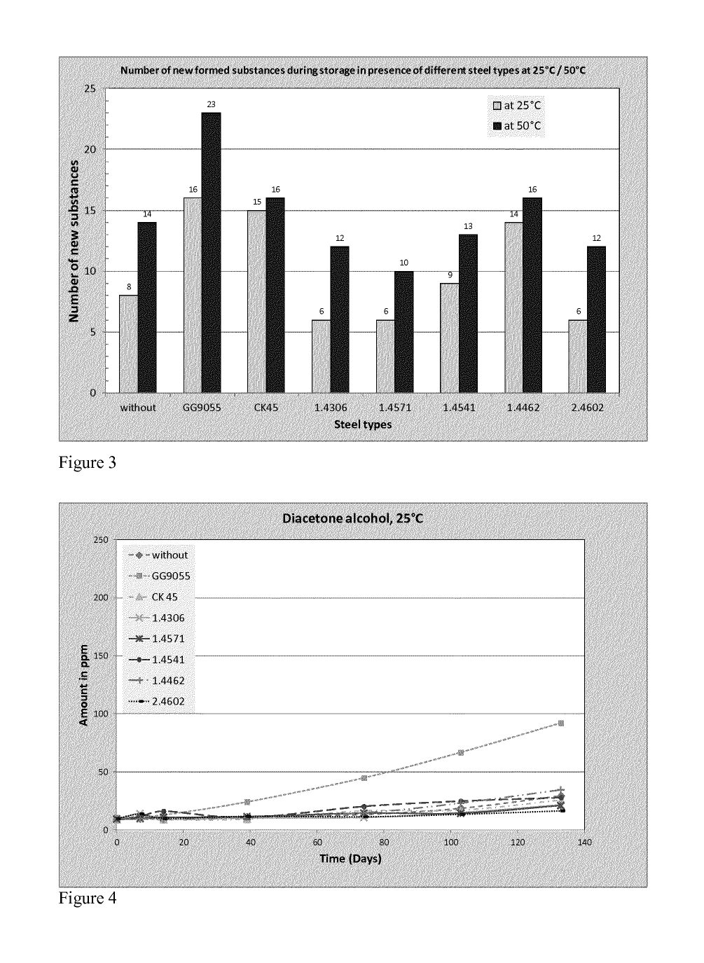

FIG. 3 shows the number of newly formed compounds on storage at these two temperatures.

FIG. 4 is an illustration of DAA formed vs time at 25° C.

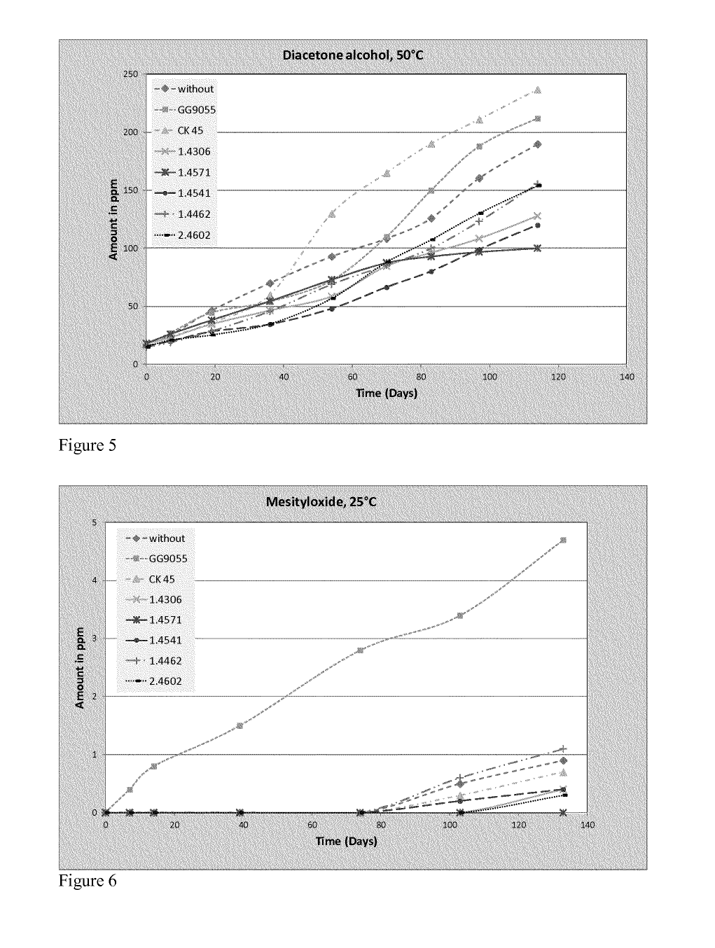

FIG. 5 is an illustration of DAA formed vs time at 50° C.

FIG. 6 is an illustration of MO formed vs time at 25° C.

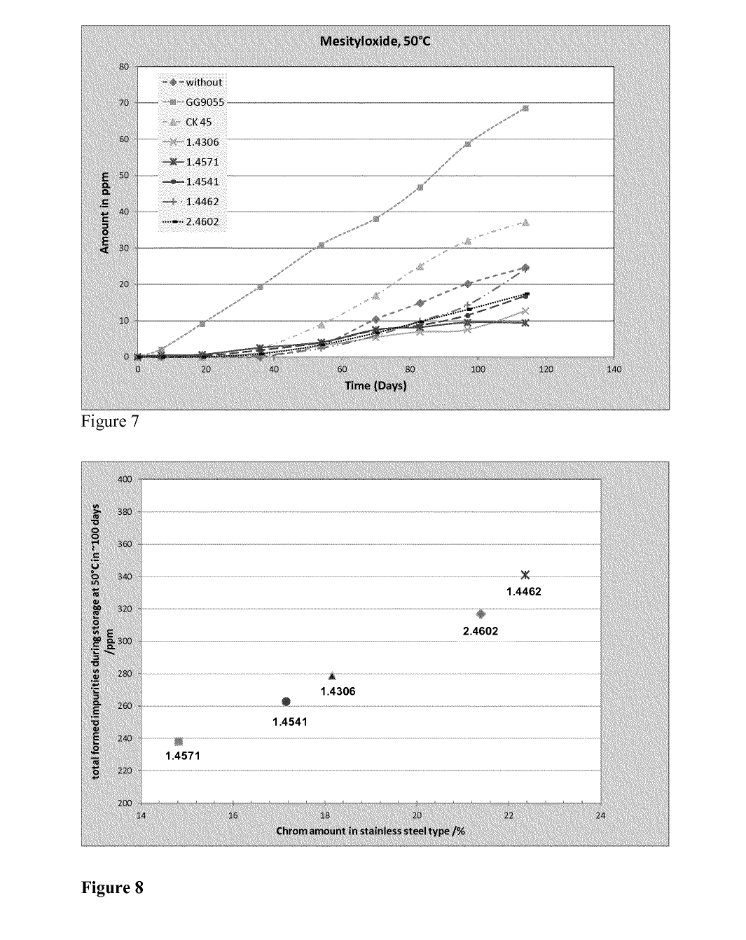

FIG. 7 is an illustration of MO formed vs time at 50° C.

FIG. 8 is an overview of the impurity formation during acetone storage in presence of different steel types.

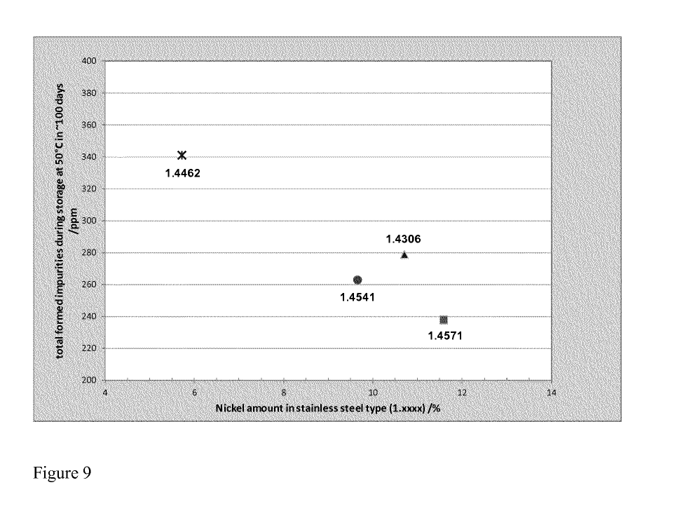

FIG. 9 is an overview of the impurity formation during acetone storage in presence of different steel types.

### EXAMPLES

Various different stainless steel bars are tested along with carbon steel and cast iron as control experiments. The storage temperatures were defined at 25° C. and 50° C. The metals were used as bars and the surface area of each bar was approximately the same. Table 1 summarises the main elemental contents of the steels employed in the examples.

TABLE 1
Used steel samples in the sample sets for
acetone storage with its chemical analysis

| | Metal Type | | | | | | |
|---|---|---|---|---|---|---|---|
| | GG 9055 | CK 45 | | | | | Hastelloy |
| elem. | Cast | Carbon | | | | | C22 - |
| Content % | iron | Steel | 1.4306 | 1.4571 | 1.4462 | 1.4541 | 2.4602 |
| C | 3.44 | 0.43 | 0.027 | 0.03 | 0.019 | 0.019 | 0.005 |
| Si | 1.77 | 0.29 | 0.56 | 0.47 | 0.4 | 0.51 | 0.026 |
| Mn | 0.47 | 0.58 | 1.34 | 1.49 | 1.51 | 1.91 | 0.26 |
| Cr | 0.02 | 0.13 | 18.15 | 14.82 | 22.36 | 17.15 | 21.39 |
| Ni | 0.18 | 0.09 | 10.7 | 11.58 | 5.72 | 9.65 | n.a. |
| Mo | 0 | 0.05 | n.a | 2.16 | 3.17 | n.a. | 13.49 |

The storage experiments were carried out in transparent glass bottles, stored in the dark. The 50° C. samples were stored in a drying oven. Every few days samples were taken out and analysed by gas chromatography. Storage in the dark was chosen to minimize influence of daylight, especially UV-light and to mimic industrial reality as acetone is stored in the dark. As a control, an acetone sample was stored in brown bottles without steel.

The GC analysis was carried out using a GC Perkin Elmer, detection with FID and as column DB-Wax 30 m 0.25 ID.

| The GC analysis of the starting | |
|---|---|
| acetone is shown below. Impurity | ppm |
| DAA | 14 |
| Mesityloxide | 0 |
| Total Impurities | 148 |

### Results

FIGS. 1 and 2 show the total amount of impurities formed in the acetone during storage over time. The highest increase occurs with cast iron (GG9950).

At 50° C. an extreme increase in the amount of impurities takes place. Starting from a total amount of 150 ppm the amount raises up to 450 ppm. The two samples with highest rise are cast iron (GG9950) and carbon steel (CK45). The best inhibition of impurities is detected with “1.4306” and “1.4571” steel grades.

Besides the six known impurities (acetaldehyde, propionaldehyde, methanol, DAA, ethanol and benzene) in all samples new peaks are formed during storage. FIG. 3 shows the number of new peaks in the GC chromatogram. At 25° C. between 6 and 15 new peaks occur, at 50° C. up to 23 new peaks. In most cases the amount is smaller than 10 ppm.

In FIG. 4 at 25° C. the time dependent formation of diacetone alcohol is shown. At 25° C. no significant rise is visible after 3 months (90 days), except for the sample with cast iron. Here DAA is continuously formed and achieves 100 ppm after 4 months. In case of the sample with 1.4541 a low increase takes place.

At 50° C. (FIG. 5) the sample without steel increases significantly from 14 to 200 ppm in 4 months. Probably the slightly alkaline pH value plays an important role. The sample CK45 also shows a high amount of DAA after 4 months. All samples with stainless steel have a lower formation rate. The best steel type here is 1.4571. All samples stored at 50° C. contain latest after 3 months more than 80 ppm DAA, which is the preferred upper limit of the specification.

FIG. 6 demonstrates the same effect for mesityloxide (MO). After a time of 75 days at 25° C. and 30 days at 50° C. (FIG. 7) the amount of mesityloxide increases to detectable amounts. At 25° C. the formation is rather slow and stays for 4 months under the preferred upper limit of 5 ppm. However, at 50° C. the limit of MO is surpassed after 70 days. The best steel type here is 1.4571, same as for DAA formation.

### Example 2

The results described above were obtained by continuous sampling of the acetone from the storage vessel. To determine whether continuous sampling was having an effect, a 2nd sample set was stored under the same conditions but without periodical sampling. The bottles were closed over 104 storage days and after opening an analysis was done. The results in comparison to the 1st sample set are shown in Table 4 (114 days=example 1). There is a small but insignificant difference in the storage days.

In case of MO in the 2nd sample set, except with GG9950, the formation of MO is approximately “zero”, although the DAA formation shows the same growth as the 1st sample set. Two possible reasons are limited oxygen content due to closed storage and no water elimination at DAA due to missing exchange of the water, because every opening of the bottles changes the humidity in the gas phase. Through this result the sum amount of the new formed impurities is lower as well and the number of impurities is also lower.

| | | | | | number of |
|---|---|---|---|---|---|
| | after 114 days | MO | DAA | Total | new formed |
| No. | (2) after 104 days¹⁾ | ppm | | | subst. |
| 2009- | starting point | 0 | 14 | 148 | 0 |
| 0085-01 | Without | 25 | 190 | 355 | 14 |
| | without (2) | 1 | 220 | 385 | 11 |
| 2009- | GG9950 | 69 | 212 | 430 | 23 |
| 0085-02 | GG9950 (2) | 70 | 195 | 413 | 13 |
| 2009- | CK45 | 37 | 237 | 406 | 16 |
| 0085-03 | CK 45 (2) | 1 | 198 | 390 | 4 |
| 2009- | 1.4306 | 13 | 128 | 280 | 12 |
| 0085-04 | 1.4306 (2) | 1 | 198 | 397 | 7 |
| 2009- | 1.4462 | 17 | 156 | 392 | 14 |
| 0085-07 | | | | | |
| 2009- | 1.4571 | 9 | 100 | 238 | 10 |
| 0085-05 | 1.4571 (2) | 1 | 190 | 389 | 7 |
| 2009- | 1.4541 | 17 | 120 | 262 | 13 |
| 0085-06 | | | | | |
| 2009- | 2.4602 | 18 | 154 | 317 | 12 |
| 0085-08 | 2.4602 (2) | 0 | 185 | 387 | 7 |

The acetone storage experiment in presence of different steel types shows depending on the storage temperature (25° C. and 50° C.) significant differences. At 25° C. the formation of the main by-product diacetone alcohol (DAA) takes place especially for cast iron (GG9950). These materials perform worse than if there is no steel at all. In all samples new impurities were formed during the storage. The highest amounts were detected in the samples with the carbon steel CK45, and cast iron (GG9950).

At 50° C. the formation of the main by-product DAA occurs. Here, the temperature rise is enough to accelerate the impurity formation. After a 2 month storage at 50° C. the concentration of DAA pass the specification of max. 80 ppm. At 50° C. the number of new formed impurities increases, especially in case of cast iron. 23 new impurities were formed.

It should be noted that Hastelloy steels (2.4602) containing large amounts of Cr and Mo are prohibitively expensive for use in the formation of large tanks. The fact that the steels of the present invention perform as well as Hastelloy steels is remarkable.

1. An acetone storage tank or acetone transfer pipe, comprising: stainless steel with an amount of Cr in the range 10.5 wt % to 20 wt %; an amount of Ni of greater than or equal to 9 wt %, and an amount of Mo that is less than or equal to 2.75% and greater than or equal to 0 wt %, of the stainless steel.

2. The acetone storage tank or acetone transfer pipe of claim 1 containing acetone.

3. The acetone storage tank or acetone transfer pipe of claim 1, wherein the content of phosphorus is equal to or higher than 0.02%.

4. The acetone storage tank or acetone transfer pipe of claim 1, wherein the content of sulphur is equal or higher than 0.02%

5. The acetone storage tank or acetone transfer pipe of claim 1, wherein the steel is doped with Cu and/or Ti and/or Nb.

6. The acetone storage tank or acetone transfer pipe of claim 1, wherein the steel comprises 11 to 18.5 wt % Cr, 0 to 2.5 wt % Mo, and 9 to 15 wt % Ni.

7. The acetone storage tank or acetone transfer pipe of claim 1, wherein the steel comprises 14 to 18.5 wt % Cr, 0.5 to 2.5 wt % Mo, and 9 to 13.5 wt % Ni.

8. The acetone storage tank or acetone transfer pipe of claim 1, wherein the steel is 1.4306, 1.4541, or 1.4571 steel.

9. The acetone storage tank or acetone transfer pipe of claim 2, wherein the level of diacetone alcohol within the tank or pipe is always below 80 ppm.

10. (canceled)

11. (canceled)

12. A process for storing acetone, comprising: putting acetone having a diacetone alcohol content of less than 25 ppm and/or a mesityl oxide content of less than 1 ppm in the acetone storage tank or acetone transfer pipe of claim 1;
storing said acetone within the tank or pipe for a period of at least one month during which time the tank or pipe is repeatedly exposed to a temperature of at least 25° C. such that the amount of diacetone alcohol in the acetone after one month is below 80 ppm and/or that the amount of mesityl oxide in the acetone after one month is below 5 ppm.

13. A process for inhibiting the formation of impurities in acetone, comprising: storing acetone in the acetone storage tank of claim 1.

14. A process for inhibiting the formation of acetone oligomers and their dehydration products in acetone, comprising: storing acetone in the of claim 1.

15. A storage tank or transfer pipe for an aldehyde or ketone, comprising: stainless steel with an amount of Cr in the range of 10.5 wt % to 20 wt %; an amount of Ni of greater than or equal to 9 wt %, and an amount of Mo that is less than or equal to 2.75% and greater than or equal to 0%, of the stainless steel.
